(19) United States
(12) Patent Application Publication
(10) Pub. No.: US 2021/0071223 A1
(43) Pub. Date: Mar. 11, 2021

(54) TWO-STEP FERMENATION PROCESS FOR PRODUCTION OF A PRODUCT

(71) Applicant: LanzaTech, Inc., Skokie, IL (US)

(72) Inventors: Rasmus Jensen, Skokie, IL (US); Michael Koepke, Skokie, IL (US); Sean Simpson, Skokie, IL (US)

(21) Appl. No.: 16/963,669

(22) PCT Filed: Jan. 23, 2019
(86) PCT No.: PCT/US2019/014794

§ 371 Date: Jul. 21, 2020

Related U.S. Application Data
(60) Provisional application No. 62/621,020, filed on Jan. 23, 2018.

(51) Int. Cl. C12P 39/00; C12P 5/02; C12P 7/28; C12P 7/42

(52) U.S. Cl. CPC C12P 39/00; C12P 7/42; C12P 7/28; C12P 5/026

(57) ABSTRACT

The invention provides genetically engineered microorganisms and methods for the production of a product by a two-step fermentation process. In particular, the present invention relates to production of an intermediate by microbial fermentation of a gaseous substrate followed by conversion of the intermediate to a final product through microbial fermentation of a carbohydrate substrate.

### CROSS REFERENCE TO RELATED APPLICATIONS

This application is the U.S. national phase of International Patent Application No. PCT/US2019/014794 filed on Jan. 23, 2019, which claims the benefit of U.S. Provisional Patent Application No. 62/621,020 filed Jan. 23, 2018, both of which are incorporated by reference in their entirety.

### FIELD OF THE INVENTION

The present invention relates to genetically engineered microorganisms and methods for the production of a product, particularly by microbial fermentation of a gaseous substrate and microbial fermentation of a carbohydrate substrate.

### DESCRIPTION OF RELATED ART

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). The majority of CO₂ comes from the burning fossil fuels to produce energy, although industrial and forestry practices also emit CO₂ into the atmosphere. Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases comprising carbon dioxide (CO₂), carbon monoxide (CO), and/or hydrogen (H₂), such as industrial waste gas or syngas, into a variety of fuels and chemicals. Recently, however, gas fermentation has emerged as an alternative platform for the biological fixation of such gases. In particular, C1-fixing microorganisms have been demonstrated to convert gases comprising CO₂, CO, CH₄, and/or H₂ into products such as ethanol and 2,3-butanediol.

Although some work has been done to engineer C1-fixing microorganisms to produce additional chemicals, progress has been hindered by several factors. For example, genetic tools for working with C1-fixing microorganisms are underdeveloped compared to those for model microorganisms such as E. coli and yeast. Furthermore, C1-fixing microorganisms may be energetically limited compared to carbohydrate-consuming microorganisms, such that they are less able to support the carbon and energy demands of heterologous enzyme pathways. Thus, although there is a strong need for microorganisms and methods for producing useful products from C1 substrates, such goals are challenging, especially at commercially-viable levels.

Although carbohydrate fermentation technology is more mature, biological processes using carbohydrate-consuming microorganisms to generate products are generally inefficient, as a large portion of carbon is converted to CO₂. Consequently, there is a need in the art for fermentation methods that not only capture carbon from CO₂ emissions but also capitalize on the comparative metabolic strengths of different biological processes to produce a product.

### SUMMARY OF THE INVENTION

It is against the above background that the present invention provides certain advantages and advancements over the prior art.

Although this invention disclosed herein is not limited to specific advantages or functionalities, the invention provides a two-step fermentation method for producing a product comprising culturing a first microorganism under conditions wherein the first microorganism ferments a first feedstock to produce an intermediate and culturing a second microorganism under conditions wherein the second microorganism ferments a second feedstock to produce the product from the intermediate.

In some aspects of the method disclosed herein, the first feedstock or the second feedstock is a gaseous substrate.

In some aspects of the method disclosed herein, the gaseous substrate comprises one or more of CO, CO₂, H₂, and CH₄.

In some aspects of the method disclosed herein, the first feedstock or the second feedstock is a carbohydrate.

In some aspects of the method disclosed herein, the carbohydrate comprises one or more of xylose, arabinose, glucose, fructose, mannose, galactose, fucose, sucrose, maltose, melibiose, xylan, xylogluco-oligosaccharides, and mannitol.

In some aspects of the method disclosed herein, the first microorganism or the second microorganism is a C1-fixing microorganism.

In some aspects of the method disclosed herein, the C1-fixing microorganism is a member of a genus selected from the group consisting of Acetobacterium, Alkalibaculum, Blautia, Butyribacterium, Clostridium, Eubacterium, Moorella, Oxobacter, Sporomusa, and Thermoanaerobacter.

In some aspects of the method disclosed herein, the C1-fixing microorganism is derived from a parental bacterium selected from the group consisting of Clostridium autoethanogenum, Clostridium ljungdahlii, and Clostridium ragsdalei.

In some aspects of the method disclosed herein, the first microorganism or the second microorganism is a carbohydrate-fermenting microorganism.

In some aspects of the method disclosed herein, the carbohydrate-fermenting microorganism is selected from the group consisting of Escherichia coli, Bacillus subtilis, Caldicellulosiruptor saccharolyticus, Clostridium acetobutylicum, Clostridium beijerinckii, Lactococcus lactis, Lactobacillus, Klebsiella, Thermoplasma acidophilum, Picrophilus torridus, Zymomonas mobilis, Saccharomyces cerevisiae, Schizosaccharomyces pombe, Schwanniomyces (Debaryomyces) occidentalis, Kluyveromyces marxianus, and Yarrowia lipolytica.

In some aspects of the method disclosed herein, the first microorganism and the second microorganism are cultured in one bioreactor.

In some aspects of the method disclosed herein, the first microorganism is cultured in a first bioreactor to produce the intermediate, and the second microorganism is cultured in a second bioreactor to produce the product from the intermediate.

In some aspects of the method disclosed herein, at least a portion of an intermediate produced in the first bioreactor is passed to the second bioreactor.

In some aspects of the method disclosed herein, the intermediate is selected from the group consisting of acetone, β-hydroxyisovaleric acid, 3-hydroxybutyrate, mevalonate, 2-oxoglutarate, a fatty acid, a carboxylic acid, a dicarboxylic acid, a hydroxy acid, and chorismate, and the product is selected from the group consisting of isobutylene, 1,3-butanediol, isoprene, an isoprenoid, succinate, an alcohol, an alkane, an alkene, a diol, and vanillin.

In some aspects of the method disclosed herein, the intermediate is acetone or β-hydroxyisovaleric acid, and the product is isobutylene.

In some aspects of the method disclosed herein, the first microorganism is a C1-fixing microorganism, first feedstock is a gaseous substrate, the second microorganism is a carbohydrate-fermenting microorganism, the second feedstock is a carbohydrate substrate, the C1-fixing microorganism ferments the gaseous substrate to produce the intermediate, and the carbohydrate-fermenting microorganism ferments the carbohydrate substrate to produce the product from the intermediate.

In some aspects of the method disclosed herein, the C1-fixing microorganism comprises one or more enzymes selected from the group consisting of an enzyme capable of converting acetyl-CoA to acetoacetyl-CoA, an enzyme capable of converting acetoacetyl-CoA to acetoacetate, and an enzyme capable of converting acetoacetate to acetone, and the carbohydrate-fermenting microorganism comprises one or more enzymes selected from the group consisting of an enzyme capable of converting acetone to β-hydroxyisovaleric acid and an enzyme capable of converting β-hydroxyisovaleric acid to isobutylene.

In some aspects of the method disclosed herein, the carbohydrate-fermenting microorganism further produces CO₂.

In some aspects of the method disclosed herein, CO₂ produced by the carbohydrate-fermenting microorganism is a substrate for a C1-fixing microorganism.

These and other features and advantages of the present invention will be more fully understood from the following detailed description taken together with the accompanying claims. It is noted that the scope of the claims is defined by the recitations therein and not by the specific discussion of features and advantages set forth in the present description.

### DESCRIPTION OF THE DRAWINGS

The following detailed description of the embodiments of the present invention can be best understood when read in conjunction with the following drawings, where like structure is indicated with like reference numerals and in which:

FIG. 1A is a schematic showing a first microorganism (organism 1) that is a C1-fixing microorganism capable of producing an intermediate from a gaseous substrate and (2) a second microorganism (organism 2) that is a carbohydrate-fermenting microorganism capable of producing a product from the intermediate produced by the first microorganism (organism 1). FIG. 1B is a schematic showing a first microorganism (organism 1) that is a carbohydrate-fermenting microorganism capable of producing an intermediate from a sugar and a second microorganism (organism 2) that is a C1-fixing microorganism capable of producing a product from the intermediate produced by the first microorganism (organism 1).

FIG. 2 is a schematic showing a two-bioreactor fermentation process.

FIG. 3A is a schematic showing (1) a C1-fixing step for the production of acetone (an intermediate) from a C1-substrate and (2) a carbon-oxidizing process that converts acetone into a final product, isobutylene. FIG. 3B is a schematic showing (1) a C1-fixing step for the production of β-hydroxyisovaleric acid (an intermediate) from a C1-substrate and (2) a carbon-oxidizing step that converts β-hydroxyisovaleric acid into a final product, isobutylene.

FIG. 4 is a schematic showing (1) a C1-fixing step for the production of mevalonate (an intermediate) from a C1-substrate and (2) a carbon-oxidizing step that converts mevalonate into a final terpene product, such as isoprene or an isoprenoid.

FIG. 5A is a schematic showing a first microorganism that is a carbohydrate-fermenting microorganism capable of producing carboxylic and/or dicarboxylic acids from a sugar and a second microorganism that is a C1-fixing microorganism capable of producing alcohols and/or diols from the carboxylic and/or dicarboxylic acids produced by the first microorganism. FIG. 5B is a schematic showing a first microorganism that is a carbohydrate-fermenting microorganism capable of producing saturated and/or unsaturated fatty acids from a sugar and a second microorganism that is a C1-fixing microorganism capable of producing saturated and/or unsaturated fatty alcohols from the saturated and/or unsaturated fatty acids produced by the first microorganism.

### DETAILED DESCRIPTION OF THE INVENTION

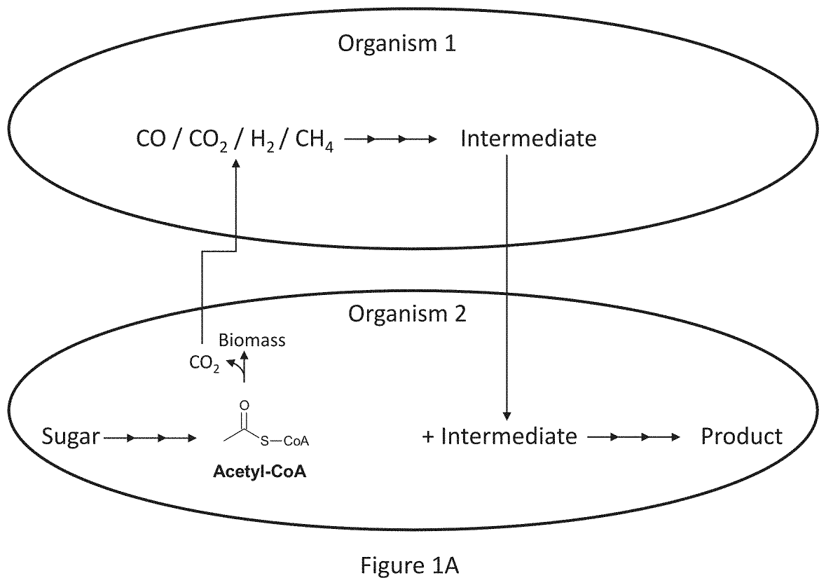

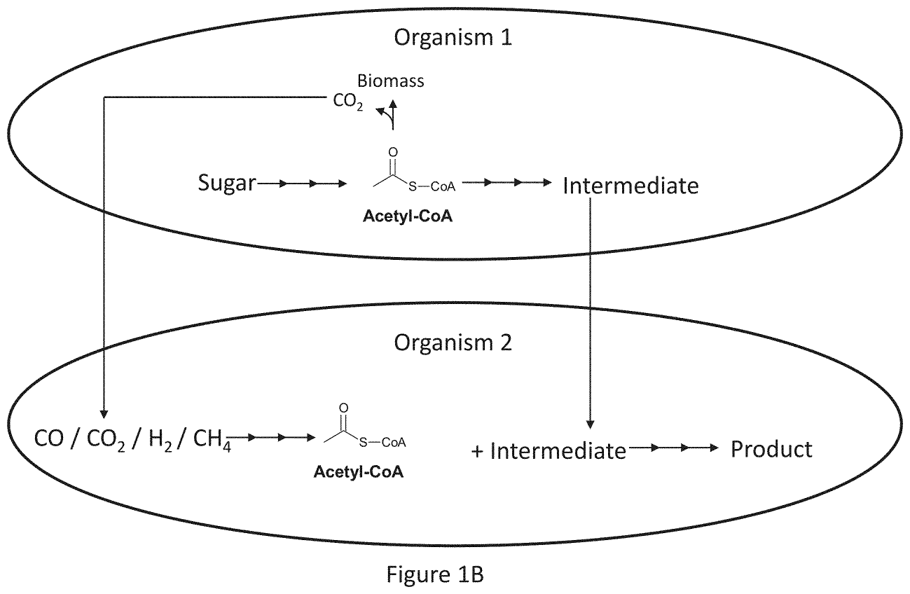

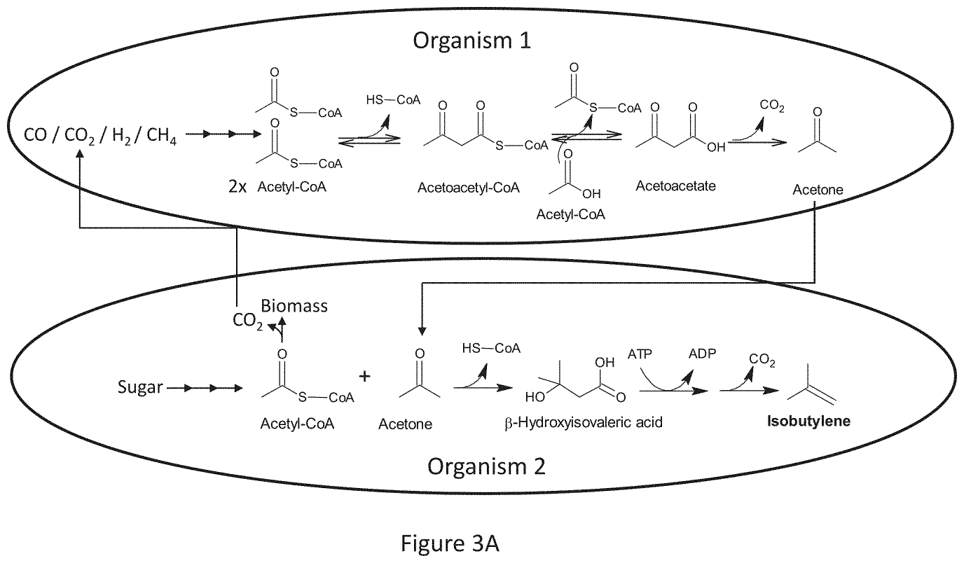

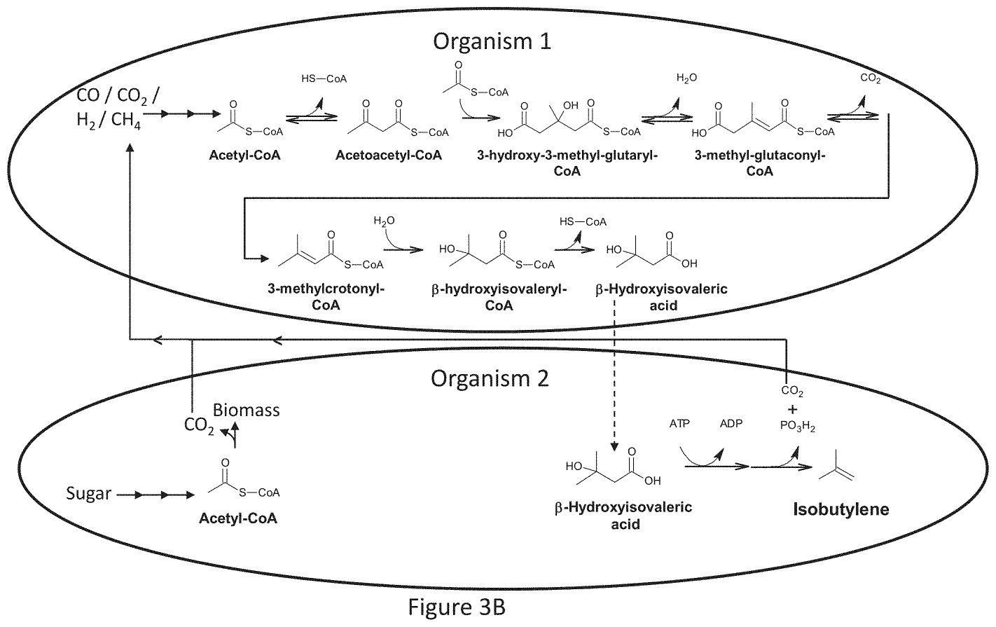

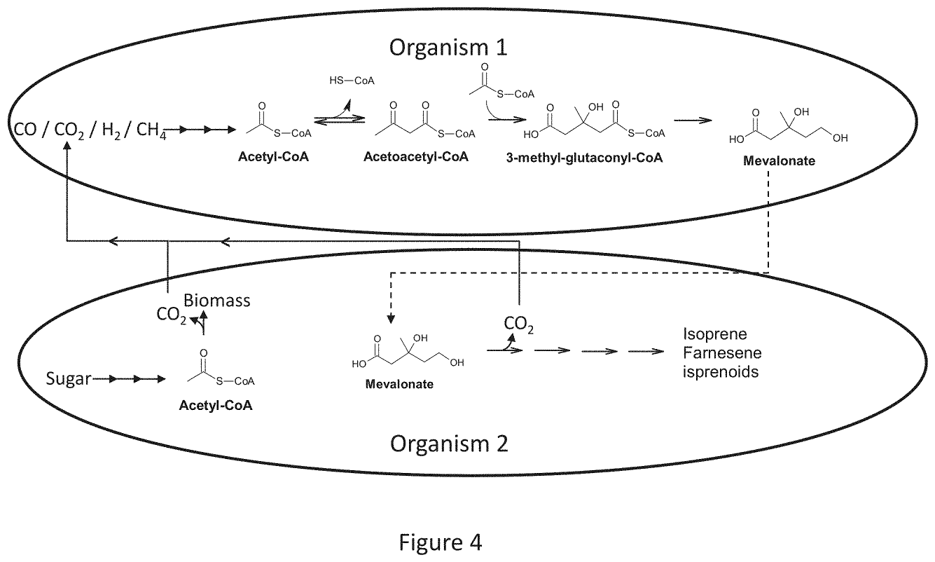

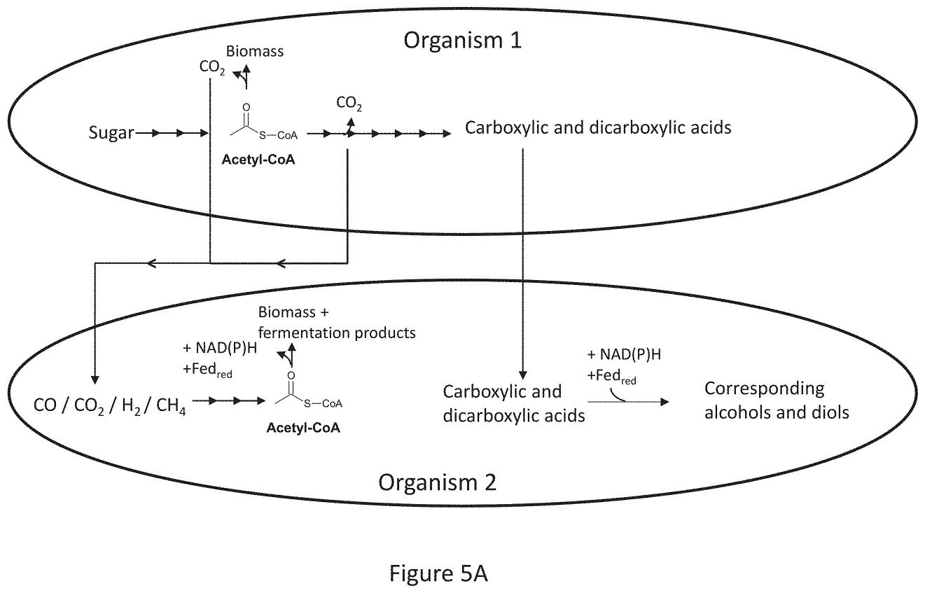

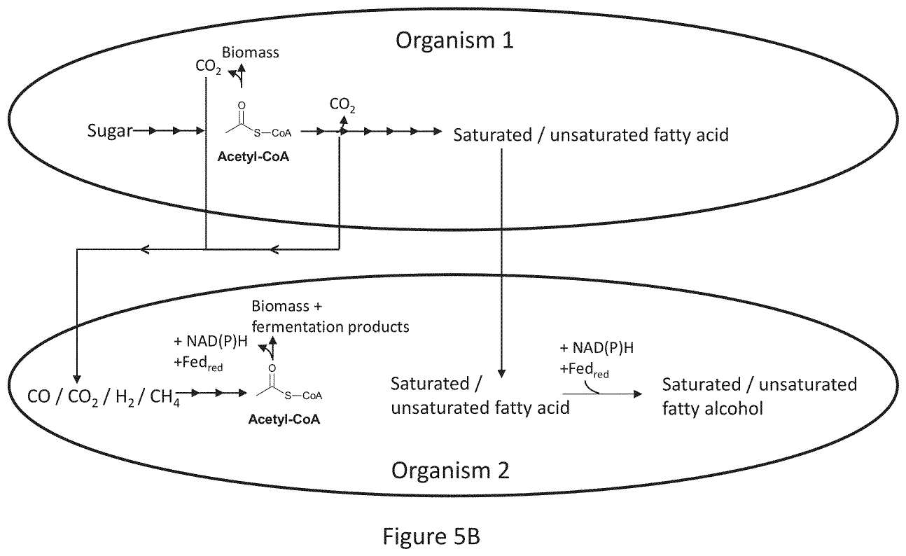

The invention provides microorganisms for the biological production of a product via an intermediate (FIGS. 1A and 1B). As described herein, the intermediate may be acetone or β-hydroxyisovaleric acid, and the product may be isobutylene (FIGS. 3A and 3B). In another non-limiting example, the intermediate may be mevalonate, and the product may be isoprene or an isoprenoid such as farnesene (FIG. 4). In yet another non-limiting example, the intermediate may be a saturated/unsaturated fatty acid or a carboxylic/dicarboxylic acid, and the product may be a saturated/unsaturated fatty alcohol or an alcohol/diol, respectively (FIGS. 5A and 5B).

Unless otherwise defined, the following terms as used throughout this specification are defined as follows:

A “microorganism” is a microscopic organism, especially a bacterium, archaeon, virus, or fungus. A microorganism of the invention is typically a bacterium. As used herein, recitation of “microorganism” should be taken to encompass “bacterium.”

The terms “genetic modification,” “genetic alteration,” or “genetic engineering” broadly refer to manipulation of the genome of a microorganism by the hand of man. Likewise, the terms “genetically modified,” “genetically altered,” or “genetically engineered” refer to a microorganism comprising a genetic modification or genetic alteration. These terms may be used to differentiate a lab-generated microorganism from a naturally-occurring microorganism. Methods of genetic modification include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

A “parental microorganism” is a microorganism used to generate a microorganism of the invention. A parental microorganism may be a naturally-occurring microorganism (i.e., a wild-type microorganism) or a microorganism that has been previously modified (i.e., a mutant or recombinant microorganism). A microorganism of the invention may be modified to express or overexpress one or more enzymes that were not expressed or overexpressed in a parental microorganism. Similarly, a microorganism of the invention may be modified to comprise one or more genes that a parental microorganism does not comprise. A microorganism of the invention may also be modified to not express or to express lower amounts of one or more enzymes that are expressed in a parental microorganism.

“Recombinant” indicates that a nucleic acid, protein, or microorganism is the product of genetic modification, engineering, or recombination. Generally, the term “recombinant” refers to a nucleic acid, protein, or microorganism that comprises or is encoded by genetic material derived from multiple sources, such as two or more different strains or species of microorganisms.

“Wild type” refers to the form of an organism, strain, gene, or characteristic as it occurs in nature, as distinguished from mutant or variant forms. The term “non-naturally occurring” when used in reference to a microorganism is intended to mean that the microorganism has at least one genetic modification not found in a naturally-occurring strain of the referenced species, including wild-type strains of the referenced species. Non-naturally occurring microorganisms are typically developed in a laboratory or research facility.

“Endogenous” refers to a nucleic acid or protein that is present or expressed in a wild-type or parental microorganism from which a microorganism of the invention is derived. For example, an endogenous gene is a gene that is natively present in a wild-type or parental microorganism from which a microorganism of the invention is derived. In one embodiment, expression of an endogenous gene may be controlled by an exogenous regulatory element, such as an exogenous promoter.

“Exogenous” refers to a nucleic acid or protein that originates outside of a microorganism of the invention. For example, an exogenous gene or enzyme may be artificially or recombinantly created and introduced into or expressed in a microorganism of the invention. An exogenous gene or enzyme may also be isolated from a heterologous microorganism and introduced into or expressed in a microorganism of the invention. Exogenous nucleic acids may be adapted to integrate into the genome of a microorganism of the invention or to remain in an extra-chromosomal state in a microorganism of the invention, for example, in a plasmid.

“Heterologous” refers to a nucleic acid or protein that is not present in the wild-type or parental microorganism from which a microorganism of the invention is derived. For example, a heterologous gene or enzyme may be derived from a different strain or species and introduced into or expressed in a microorganism of the invention. The heterologous gene or enzyme may be introduced into or expressed in a microorganism of the invention in the form in which it occurs naturally. Alternatively, the heterologous gene or enzyme may be modified in some way, e.g., by codon-optimization for expression in a microorganism of the invention or by engineering to have an altered function or altered substrate specificity.

The terms “nucleic acid,” “nucleotide,” “nucleotide sequence,” “oligonucleotide,” and “polynucleotide” are used interchangeably to refer to a polymeric form of nucleotides of any length, either deoxyribonucleotides or ribonucleotides, or analogs thereof. Polynucleotides may have any three-dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. The sequence of nucleotides may be interrupted by non-nucleotide components. If present, modifications to the nucleotide structure may be imparted before or after assembly of the polymer, such as by conjugation with a labeling component.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene products.”

“Enzyme activity,” and simply “activity,” refer broadly to enzymatic activity, including, but not limited, to catalytic activity of an enzyme, an amount of an active enzyme, or the availability of an enzyme to catalyze a reaction. Accordingly, “increasing” enzyme activity includes increasing the activity of an enzyme, increasing the amount of an active enzyme, or increasing the availability of an enzyme to catalyze a reaction. Similarly, “decreasing” enzyme activity includes decreasing the activity of an enzyme, decreasing the amount of an active enzyme, or decreasing the availability of an enzyme to catalyze a reaction.

“Overexpressed” refers to an increase in expression of a nucleic acid or protein in a microorganism of the invention compared to the wild-type or parental microorganism from which a microorganism of the invention is derived. Overexpression may be achieved by any means known in the art, including by modifying gene copy number, gene transcription rate, gene translation rate, or enzyme degradation rate.

“Mutated” refers to a nucleic acid or protein that has been modified in a microorganism of the invention compared to the wild-type or parental microorganism from which a microorganism of the invention is derived. In one embodiment, the mutation may be a deletion, insertion, or substitution in a gene encoding an enzyme. In another embodiment, the mutation may be a deletion, insertion, or substitution of one or more amino acids in an enzyme.

The terms “codon optimization” and “codon adaptation” refer to the mutation of a nucleic acid, such as a gene, for optimized or improved expression in a particular strain or species. Codon optimization may result in faster translation rates or higher translation accuracy. In a preferred embodiment, genes of the invention are codon optimized for expression in Clostridium, particularly Clostridium autoethanogenum, Clostridium ljungdahlii, or Clostridium ragsdalei. In a further preferred embodiment, genes of the invention are codon optimized for expression in Clostridium autoethanogenum LZ1561, which is deposited under DSMZ accession number DSM23693.

The term “variant” includes a nucleic acid or protein sequence that varies from the sequence of a reference nucleic acid and protein, such as a nucleic acid or protein disclosed in the public domain. The invention may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as GenBank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

The term “derived from” indicates that a nucleic acid, protein, or microorganism is modified or adapted from a different (e.g., a parental or wild-type) nucleic acid, protein, or microorganism so as to produce a new nucleic acid, protein, or microorganism. Such modifications or adaptations typically include insertion, deletion, mutation, or substitution of nucleic acids or genes. Generally, a microorganism of the invention is derived from a parental microorganism.

Nucleic acids may be delivered to a microorganism of the invention using any method known in the art. For example, nucleic acids may be delivered as naked nucleic acids or may be formulated with one or more agents, such as liposomes. The nucleic acids may be DNA, RNA, cDNA, or combinations thereof. Delivery vectors may include plasmids, viruses, bacteriophages, cosmids, and artificial chromosomes. In a preferred embodiment, nucleic acids are delivered to a microorganism of the invention using a plasmid. By way of example, transformation (including transduction or transfection) may be achieved by electroporation, ultrasonication, polyethylene glycol-mediated transformation, chemical or natural competence, protoplast transformation, prophage induction, or conjugation. Restriction inhibitors may be used in certain embodiments. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter.

Methods well known to those skilled in the art can be used to construct genetic expression constructs and recombinant cells according to this invention. These methods include in vitro recombinant DNA techniques, synthetic techniques, and in vivo recombination techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York.

The term “fermentation” should be interpreted as a metabolic process that produces chemical changes in a substrate. For example, a fermentation process receives one or more substrates and produces one or more products through utilization of one or more microorganisms. Preferably, a fermentation process includes the use of one or more bioreactors. The fermentation process may be described as either “batch” or “continuous.” “Batch fermentation” is used to describe a fermentation process wherein a bioreactor is filled with a raw material, e.g. a carbon source, along with a microorganism, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time and product and/or metabolites are extracted during fermentation. Preferably, the fermentation process is continuous.

“Substrate” refers to a carbon and/or energy source for a microorganism of the invention. In some embodiments, the substrate is gaseous and comprises a C1-carbon source, for example, CO, CO₂, and/or CH₄. Preferably, the substrate comprises a C1-carbon source of CO or CO and CO₂. The gaseous substrate may further comprise other non-carbon components, such as H₂, N₂, or electrons. In other embodiments, the substrate is a carbohydrate such as sugar, starch, lignin, cellulose, or hemicellulose.

The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors.

As used herein, the terms “culture” and “fermentation” are used interchangeably. These terms encompass both the growth phase and product biosynthesis phase of a fermentation process. The culture is generally maintained in an aqueous culture medium that comprises nutrients, vitamins, and/or minerals sufficient to permit growth of a microorganism. Preferably, the aqueous culture medium is an anaerobic microbial growth medium, such as a minimal anaerobic microbial growth medium. Suitable media are well known in the art.

As used herein, the terms “fermentation broth” or “broth” refer to the mixture of components in a bioreactor, which includes cells and nutrient media. As used herein, a “separator” is a module that is adapted to receive fermentation broth from a bioreactor and pass the broth through a filter to yield a “retentate” and a “permeate.” The filter may be a membrane, e.g. a cross-flow membrane or a hollow fibre membrane. The term “permeate” is used to refer to substantially soluble components of the broth that pass through the separator. The permeate will typically contain soluble fermentation products, byproducts, and nutrients. The retentate will typically contain cells. As used herein, the term “broth bleed” is used to refer to a portion of the fermentation broth that is removed from a bioreactor and not passed to a separator.

A “native product” is a product produced by a wild-type microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a wild-type microorganism from which the genetically modified microorganism is derived.

As used herein, the terms “intermediate” and “precursor” can be used interchangeably to refer to a substance, such as a molecule, compound, or protein, that is produced upstream of a particular product. The intermediate may be directly upstream of the product. The intermediate may be indirectly upstream of the product. For example, in the exemplary reaction “compound A”→“compound B”→“compound C”→“compound D”, “compound” C is an intermediate that is directly upstream of the product, “compound D,” and “compound B” is an intermediate that is indirectly upstream of the product, “compound D.”

“Selectivity” refers to the ratio of the production of a target product to the production of all fermentation products produced by a microorganism. A microorganism of the invention may be engineered to produce products at a certain selectivity or at a minimum selectivity. In one embodiment, a target product accounts for at least about 5%, 10%, 15%, 20%, 30%, 50%, or 75% of all fermentation products produced by a microorganism of the invention. In one embodiment, the target product accounts for at least 10% of all fermentation products produced by a microorganism of the invention, such that a microorganism of the invention has a selectivity for the target product of at least 10%. In another embodiment, the target product accounts for at least 30% of all fermentation products produced by a microorganism of the invention, such that a microorganism of the invention has a selectivity for the target product of at least 30%.

A fermentation should desirably be carried out under appropriate conditions for production of a target product. Typically, a fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, and maximum product concentrations to avoid product inhibition.

If a substrate is a gaseous substrate, maximum gas substrate concentrations should be considered to ensure that gas in the liquid phase does not become limiting since products may be consumed by the culture under gas-limited conditions. Operating a bioreactor at elevated pressures allows for an increased rate of gas mass transfer from the gas phase to the liquid phase. Also, since a given gas conversion rate is, in part, a function of the substrate retention time, and retention time dictates the required volume of a bioreactor, the use of pressurized systems can greatly reduce the volume of the bioreactor required and, consequently, the capital cost of the fermentation equipment. This, in turn, means that the retention time, defined as the liquid volume in the bioreactor divided by the input gas flow rate, can be reduced when bioreactors are maintained at elevated pressure rather than at atmospheric pressure.

The optimum reaction conditions will depend partly on the particular microorganism used. However, in general, it is preferable to operate the fermentation at a pressure higher than atmospheric pressure. In certain embodiments, the fermentation is performed in the absence of light or in the presence of an amount of light insufficient to meet the energetic requirements of photosynthetic microorganisms. In certain embodiments, a microorganism of the invention is a non-photosynthetic microorganism.

“Increasing the efficiency,” “increased efficiency,” and the like include, but are not limited to, increasing growth rate, product production rate or volume, product volume per volume of substrate consumed, or product selectivity. Efficiency may be measured relative to the performance of a parental microorganism from which a microorganism of the invention is derived. The terms “increasing the efficiency”, “increased efficiency” and the like, when used in relation to a fermentation process, include, but are not limited to, increasing one or more of the rate of growth of microorganisms catalyzing the fermentation, the growth and/or product production rate at elevated product concentrations, increasing the volume of desired product produced per volume of substrate consumed, increasing the rate of production or level of production of the desired product, increasing the relative proportion of the desired product produced compared with other by-products of the fermentation, decreasing the amount of water consumed by the process, and decreasing the amount of energy utilized by the process.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably recycled back to the bioreactor. The cell-free permeate remaining after target products have been removed is also preferably returned to the bioreactor. Additional nutrients (such as B vitamins) may be added to the cell-free permeate to replenish the medium before it is returned to the bioreactor.

### Gas Fermentation

The term “gas fermentation” should be interpreted as a process that receives one or more gas substrates, such as industrial waste gas or syngas produced by gasification, and produces one or more product through the utilization of one or more C1-fixing microorganisms.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for a microorganism of the invention. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1 carbon source.

A microorganism of the invention may be further classified based on functional characteristics. For example, a microorganism of the invention may be or may be derived from a C1-fixing microorganism, an anaerobe, an acetogen, an ethanologen, a carboxydotroph, and/or a methanotroph. Table 1 provides a representative list of microorganisms and identifies their functional characteristics. In a preferred embodiment, a microorganism of the invention is derived from a C1-fixing microorganism identified in Table 1.

TABLE 1

| | Wood- | C1- | | | | | |
|---|---|---|---|---|---|---|---|
| | Ljungdahl | fixing | Anaerobe | Acetogen | Ethanologen | Autotroph | Carboxydotroph |
| Acetobacterium woodii | + | + | + | + | +/− ¹ | + | − |
| Alkalibaculum bacchii | + | + | + | + | + | + | + |
| Blautia producta | + | + | + | + | − | + | + |
| Butyribacterium | + | + | + | + | + | + | + |
| methylotrophicum | | | | | | | |
| Clostridium aceticum | + | + | + | + | − | + | + |
| Clostridium | + | + | + | + | + | + | + |
| autoethanogenum | | | | | | | |
| Clostridium | + | + | + | + | + | + | + |
| carboxidivorans | | | | | | | |
| Clostridium coskatii | + | + | + | + | + | + | + |
| Clostridium drakei | + | + | + | + | − | + | + |
| Clostridium | + | + | + | + | − | + | + |
| formicoaceticum | | | | | | | |
| Clostridium | + | + | + | + | + | + | + |
| ljungdahlii | | | | | | | |
| Clostridium magnum | + | + | + | + | − | + | +/− ² |
| Clostridium ragsdalei | + | + | + | + | + | + | + |
| Clostridium scatologenes | + | + | + | + | − | + | + |
| Eubacterium limosum | + | + | + | + | − | + | + |
| Moorella | + | + | + | + | + | + | + |
| thermautotrophica | | | | | | | |
| Moorella thermoacetica | + | + | + | + | − ³ | + | + |
| (formerly Clostridium | | | | | | | |
| thermoaceticum) | | | | | | | |
| Oxobacter pfennigii | + | + | + | + | − | + | + |
| Sporomusa ovata | + | + | + | + | − | + | +/− ⁴ |
| Sporomusa silvacetica | + | + | + | + | − | + | +/− ⁵ |
| Sporomusa sphaeroides | + | + | + | + | − | + | +/− ⁶ |
| Thermoanaerobacter | + | + | + | + | − | + | − |
| kiuvi | | | | | | | |

¹ Acetobacterium woodi can produce ethanol from fructose, but not from gas.
² It has not been investigated whether Clostridium magnum can grow on CO.
³ One strain of Moorella thermoacetica, Moorella sp. HUC22-1, has been reported to produce ethanol from gas.
⁴ It has not been investigated whether Sporomusa ovata can grow on CO.
⁵ It has not been investigated whether Sporomusa silvacetica can grow on CO.
⁶ It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganism” refers to a microorganism comprising the Wood-Ljungdahl pathway. Generally, a microorganism of the invention comprises a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it continues to function to convert CO, CO₂, and/or H₂ to acetyl-CoA.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, a microorganism of the invention is an anaerobe. In a preferred embodiment, a microorganism of the invention is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3rd edition, p. 354, New York, N.Y., 2006). All naturally occurring acetogens are C1-fixing, anaerobic, autotrophic, and non-methanotrophic. Typically, a microorganism of the invention is an acetogen. In a preferred embodiment, a microorganism of the invention is derived from an acetogen identified in Table 1.

An “ethanologen” is a microorganism that produces or is capable of producing ethanol. Typically, a microorganism of the invention is an ethanologen. In a preferred embodiment, a microorganism of the invention is derived from an ethanologen identified in Table 1.

An “autotroph” is a microorganism capable of growing in the absence of organic carbon. Instead, autotrophs use inorganic carbon sources, such as CO and/or CO₂. Typically, a microorganism of the invention is an autotroph. In a preferred embodiment, a microorganism of the invention is derived from an autotroph identified in Table 1.

A “carboxydotroph” is a microorganism capable of utilizing CO as a sole source of carbon and energy. Typically, a microorganism of the invention is a carboxydotroph. In a preferred embodiment, a microorganism of the invention is derived from a carboxydotroph identified in Table 1.

A “methanotroph” is a microorganism capable of utilizing methane as a sole source of carbon and energy. In certain embodiments, a microorganism of the invention is a methanotroph or is derived from a methanotroph. In other embodiments, a microorganism of the invention is not a methanotroph or is not derived from a methanotroph.

In one embodiment, a C1-fixing microorganism is Clostridium autoethanogenum, Clostridium ljungdahlii, or Clostridium ragsdalei or derived from Clostridium autoethanogenum, Clostridium ljungdahlii, or Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These three species have many similarities. In particular, these species are all C1-fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH growth ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-containing gases, produce ethanol and acetate (or acetic acid) as main fermentation products, and produce small amounts of 2,3-butanediol and lactic acid under certain conditions.

However, these three species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Kopke, Curr Opin Biotechnol, 22: 320-325, 2011).

Thus, in summary, many of the characteristics of Clostridium autoethanogenum, Clostridium ljungdahlii, or Clostridium ragsdalei are not specific to that species, but are rather general characteristics for this cluster of C1 fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

A microorganism of the invention may also be derived from an isolate or mutant of Clostridium autoethanogenum, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LBS1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

In a preferred embodiment, a parental microorganism is Clostridium autoethanogenum LZ1561, which was deposited on Jun. 7, 2010 with Deutsche Sammlung von Mikroorganismen and Zellkulturen GmbH (DSMZ) located at Inhoffenstraß 7B, D-38124 Braunschwieg, Germany on Jun. 7, 2010 under the terms of the Budapest Treaty and accorded accession number DSM23693. This strain is described in International Patent Application No. PCT/NZ2011/000144, which published as WO 2012/015317.

More broadly, a microorganism of the invention may be derived from any genus or species identified in Table 1. For example, a microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, a microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

The composition of a gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

The term “desired composition” is used to refer to the desired level and types of components in a substance, such as, for example, of a gas stream, including but not limited to syngas. More particularly, a gas is considered to have a “desired composition” if it contains a particular component (e.g., CO, H₂, and/or CO₂) and/or contains a particular component at a particular proportion and/or does not contain a particular component (e.g., a contaminant harmful to a microorganism) and/or does not contain a particular component at a particular proportion. More than one component may be considered when determining whether a gas stream has a desired composition.

A gaseous substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. A microorganism of the invention typically converts at least a portion of the CO in the substrate to a product. In some embodiments, the substrate comprises no or substantially no (<1 mol %) CO.

A gaseous substrate may comprise some amount of H₂. For example, the substrate may comprise about 1, 2, 5, 10, 15, 20, or 30 mol % H₂. In some embodiments, the substrate may comprise a relatively high amount of H₂, such as about 60, 70, 80, or 90 mol % H₂. In further embodiments, the substrate comprises no or substantially no (<1 mol %) H₂.

A gaseous substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂.

The term “carbon capture” as used herein refers to the sequestration of carbon compounds including CO₂ and/or CO from a stream comprising CO₂ and/or CO and either a) converting the CO₂ and/or CO into products, b) converting the CO₂ and/or CO into substances suitable for long-term storage, c) trapping the CO₂ and/or CO in substances suitable for long-term storage, or d) a combination of these processes.

A microorganism of the invention may be cultured with a gas stream to produce one or more products. For instance, a microorganism of the invention may produce or may be engineered to produce ethanol (WO 2007/117157), acetate (WO 2007/117157), or 2,3-butanediol (WO 2009/151342 and WO 2016/094334). In a preferred embodiment, a microorganism of the invention may produce or be engineered to produce acetone (WO 2012/115527) and/or butene (WO 2012/024522). In other embodiments, a microorganism of the invention may produce or may be engineered to produce butanol (WO 2008/115080 and WO 2012/053905), butyrate (WO 2008/115080), lactate (WO 2011/112103), butadiene (WO 2012/024522), methyl ethyl ketone (2-butanone) (WO 2012/024522 and WO 2013/185123), ethylene (WO 2012/026833), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1 propanol (WO 2014/0369152), chorismate-derived products (WO 2016/191625), 3 hydroxybutyrate (WO 2017/066498), and 1,3-butanediol (WO 2017/0066498). In certain embodiments, microbial biomass itself may be considered a product. These products, such as ethanol, may be further converted to produce at least one component of diesel, jet fuel, and/or gasoline. Additionally, the microbial biomass may be further processed to produce a single cell protein (SCP).

### Carbohydrate Fermentation

Whereas gas fermentation is a developing field, fermentation of carbohydrates has long been recognized as beneficial for the production of products. See, e.g., Müller, Encyclopedia of Life Sciences, 2001; Jones & Woods, Microbiological Reviews 50(4):484-524, 1986; Johnson & Echavarri-Erasun, Chapter 3, The Yeasts, 2014. The use of carbohydrates as a feedstock has the major advantage of generating copious amounts of energy in the form of ATP. For example, using glucose as a feedstock for aerobic fermentation, a total of 38 ATP molecules are generated. Under anaerobic conditions, 4 to 36 ATP molecules can be generated per glucose molecule, depending on the microorganism used and the operating conditions. The ATP generating properties of carbohydrate fermentations make these fermentations ideal for production of products using an energy intensive pathway. Exemplary ATP-intensive products include isoprenoids and aromatics, especially those of the shikimate pathway.

These ATP-generating properties are the result of carbon oxidation, which generates the reducing equivalents required to lower the oxygen content of an initial molecule (e.g., glucose) to a more reduced product (e.g., ethanol.) Disadvantageously, this carbon oxidation also results in a loss of carbon in the form of CO₂. Using glucose oxidation to ethanol as an example, nearly half of the glucose feedstock is lost as CO₂. As such, biological processes utilizing carbohydrate feedstocks for the production of products have a low carbon conversion efficiency.

As used herein, the term “carbohydrate-fermenting microorganism” refers to a microorganism capable of fermenting a carbohydrate such as a sugar. A carbohydrate-fermenting microorganism produces an acid or an acid with a gas when fermenting a carbohydrate. Products of carbohydrate fermentation may include lactic acid, formic acid, acetic acid, butyric acid, butyl alcohol, acetone, ethyl alcohol, CO₂, and H₂.

In some embodiments, a carbohydrate-fermenting microorganism is a bacterium, such as Escherichia coli, Bacillus subtilis, Caldicellulosiruptor saccharolyticus, Clostridium acetobutylicum, Clostridium beijerinckii, Lactococcus lactis, Lactobacillus, Klebsiella, Thermoplasma acidophilum, Picrophilus torridus, Zymomonas mobilis. In some embodiments, a carbohydrate-fermenting microorganism is a yeast, such as Saccharomyces cerevisiae, Schizosaccharomyces pombe, Schwanniomyces (Debaryomyces) occidentalis, Kluyveromyces marxianus, or Yarrowia lipolytica. Although some of the microorganisms listed in Table 1 may be capable of consuming carbohydrates, for the purposes of the present invention, the carbohydrate-fermenting microorganism is not any of the microorganisms listed in Table 1. Typically, the carbohydrate-fermenting microorganism is not capable of consuming gaseous substrates comprising, e.g., CO, CO₂, H₂, and/or CH₄.

In some embodiments, a carbohydrate feedstock comprises a monosaccharide, disaccharide, polysaccharide, and/or polyol. In some embodiments, the monosaccharide is a pentose or a hexose. In some embodiments, the pentose is xylose or arabinose. In some embodiments, the hexose is glucose, fructose, mannose, galactose, or fucose. In some embodiments, the disaccharide is sucrose, maltose, or melibiose. In some embodiments, the polysaccharide is xylan or xylogluco-oligosaccharides. In some embodiments, the polyol is mannitol.

In some embodiments, the carbohydrate-fermenting microorganism is Escherichia coli, and the carbohydrate feedstock is glucose, xylose, arabinose, fucose, galactose, mannose, or a combination thereof. In some embodiments, the carbohydrate-fermenting microorganism is Bacillus subtilis, and the carbohydrate feedstock is glucose, xylose, arabinose, fructose, sucrose, lactose, mannose, maltose, or a combination thereof. In some embodiments, the carbohydrate-fermenting microorganism is Zymomonas mobilis, and the carbohydrate feedstock is glucose, xylose, arabinose, fructose, sucrose, mannose, or a combination thereof. In some embodiments, the carbohydrate-fermenting microorganism is Clostridium acetobutylicum or Clostridium beijerinckii, and the carbohydrate feedstock is glucose, xylose, arabinose, fructose, or a combination thereof. In some embodiments, the carbohydrate-fermenting microorganism is Lactococcus lactis, and the carbohydrate feedstock is glucose, galactose, lactose, melibiose, or a combination thereof. In some embodiments, the carbohydrate-fermenting microorganism is Caldicellulosiruptor saccharolyticus, and the carbohydrate feedstock is arabinose, fructose, galactose, glucose, mannose, xylose, xylan, xylogluco-oligosaccharides, or a combination thereof. In some embodiments, the carbohydrate-fermenting microorganism is Saccharomyces cerevisiae, and the carbohydrate feedstock is glucose, mannose, galactose, or a combination thereof. In some embodiments, the carbohydrate-fermenting microorganism is Schizosaccharomyces pombe, and the carbohydrate feedstock is glucose, fructose, xylose, arabinose, sucrose, or a combination thereof. In some embodiments, the carbohydrate-fermenting microorganism is Kluyveromyces marxianus, and the carbohydrate feedstock is xylose, maltose, lactose, glucose, sucrose, mannitol, arabinose, galactose, or a combination thereof. In some embodiments, the carbohydrate-fermenting microorganism is Yarrowia lipolytica, and the carbohydrate feedstock is glucose, fructose, mannose, or a combination thereof.

The carbohydrate-fermenting microorganism is cultured in minimal defined media of a particular pH and at a defined temperature for a particular period of time. For example, the carbohydrate-fermenting microorganism may be cultured at a temperature of about 15° C. to about 80° C. In some embodiments, the carbohydrate-fermenting microorganism is cultured at 37° C. The carbohydrate-fermenting microorganism may be cultured at a pH of about 0-9, such as a pH of about 3.5-8.5.

### Two-Step Fermentation Process

The inventors have learned that production of a product can be improved by identifying and utilizing a suitable combination of fermentation processes based on their comparative metabolic strengths. In one aspect, there is provided a process having enhanced production efficiency, the process comprising (i) providing a first feedstock to a first microorganism and culturing the microorganism under conditions to produce an intermediate and (ii) providing a second feedstock to a second microorganism and culturing the microorganism under conditions to convert the intermediate to a product. As used herein, production of an intermediate as described in (i) is referred to as “step 1,” and conversion of the intermediate to produce a product as described in (ii) is referred to as “step 2.” The product produced by step 2 may also be referred to as a “final product” herein.

In some embodiments, the first feedstock is a gaseous substrate, such as a C1 feedstock, and the first microorganism is a C1-fixing microorganism (e.g., for step 1). In some embodiments, the second feedstock is a carbohydrate, and the second microorganism is a carbohydrate-fermenting microorganism (e.g., for step 2). See, e.g., FIGS. 1A, 3A, 3B, and 4.

In an alternative embodiment, the first feedstock is a carbohydrate, and the first microorganism is a carbohydrate-fermenting microorganism (e.g., for step 1). In another alternate embodiment, the second feedstock is a gaseous C1 feedstock, and the second microorganism is a C1-fixing microorganism (e.g., for step 2). See, e.g., FIGS. 1B, 5A, and 5B.

In some embodiments, step 1 and step 2 occur in a single bioreactor. In some embodiments, the first feedstock and second feedstock are provided to a bioreactor comprising a co-culture of a first microorganism and a second microorganism. In some embodiments, the co-culture comprises a C1-fixing microorganism and a carbohydrate-fermenting microorganism. In some embodiments, the first feedstock, a gaseous C1 feedstock, is converted to an intermediate by the C1-fixing microorganism, and the second feedstock, a carbohydrate feedstock, is consumed by the carbohydrate-fermenting microorganism to convert the intermediate to a final product.

In some embodiments, step 1 occurs in a first bioreactor, and step 2 occurs in a second bioreactor. For example, the first bioreactor may comprise a C1-fixing microorganism and a gaseous C1 feedstock, and the second bioreactor may comprise a carbohydrate-fermenting microorganism and a carbohydrate feedstock. In some embodiments, at least a portion of the intermediate produced by step 1 is recovered from the first bioreactor and transferred to the second bioreactor, where the intermediate is converted to a final product by the second microorganism. In some embodiments, the permeate from step 1 (media without cells) is transferred from the first bioreactor to the second bioreactor. In other embodiments, a portion or all of the contents of the first bioreactor (media with cells) is transferred to the second bioreactor comprising the second microorganism supplied with the second feedstock.

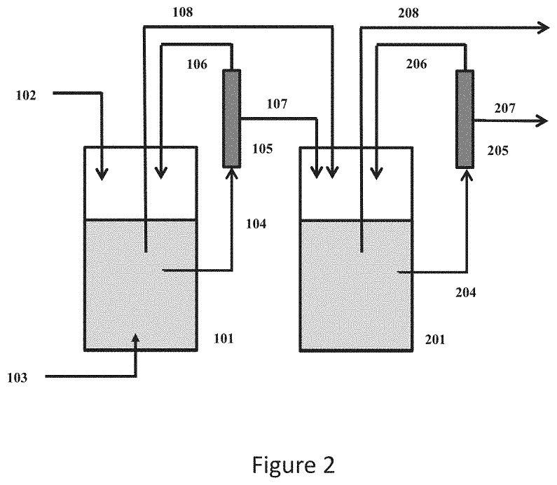

FIG. 2 shows an exemplary two bioreactor system. For step 1, the system provides a first bioreactor 101 having a media inlet 102, a gas inlet port 103, a separator means 104, a permeate stream outlet 107, and a bleed stream outlet 108. For step 2, the first bioreactor is connected to a second bioreactor 201, having a separator 205, a permeate stream outlet 207, and a bleed stream outlet 208.

As shown in FIG. 2, bioreactor 101 comprises fermentation broth comprising a culture of a first microorganism, such as a C1-fixing microorganism. Media is added to bioreactor 101 in a continuous or semi-continuous manner throughout the media inlet 102. A gaseous substrate is supplied to bioreactor 101 via the gas inlet port 103. The separator means is adapted to receive at least a portion of broth from bioreactor 101 via a first output conduit 104 and pass it through the separator 105 configured to substantially separate the microorganism cells (the retentate) from the rest of the fermentation broth (the permeate). At least a portion of the retentate is returned to bioreactor 101 via a first return conduit 106, which ensures that the broth culture density is maintained at an optimal level. The separator 105 is adapted to pass at least a portion of the permeate out of bioreactor 101 via a permeate delivery conduit 107. The permeate delivery conduit feeds the cell free permeate to bioreactor 201. In certain embodiments of the invention, at least a portion of the cell-free permeate is removed for product extraction or be recycled prior to the permeate stream being fed to bioreactor 201. A broth bleed output 108 is provided to directly feed broth from bioreactor 101 to bioreactor 201. In certain embodiments, the broth bleed and permeate bleed are combined prior to being fed to the second bioreactor. It may be desirable to purify the stream prior to passing to the second bioreactor to ensure a carbon:nitrogen ratio of at least 10:1, or at least 25:1, or at least 49:1.

The second bioreactor 201 comprises a culture of a second microorganism, such as a carbohydrate-fermenting microorganism, in a liquid nutrient medium. The second bioreactor receives broth and permeates from the first bioreactor in a continuous or semi-continuous manner through broth bleed output 108 and permeate delivery conduit 107. The separator means is adapted to receive at least a portion of broth from the bioreactor 201 via a first output conduit 204 and pass it through the separator 205 configured to substantially separate the microorganism cells (the retentate) from the rest of the fermentation broth (the permeate). At least a portion of the retentate is returned to the second bioreactor via a second return conduit 206, which ensures that the broth culture density is maintained at an optimal level. The separator 205 is adapted to pass at least a portion of the permeate out of the bioreactor 201 via a permeate removal conduit 207. A broth bleed output 208 is provided to directly remove broth from bioreactor 201. The broth bleed stream is treated to remove the biomass for lipid extraction using known methods. The substantially biomass free bleed stream and the permeate streams are combined to produce a combined stream. In certain aspects of the invention, the combined stream can be returned to the first bioreactor to supplement the liquid nutrient medium being continuously added. In certain embodiments, it may be desirable to further process the recycle stream to remove un-desired by-products of the fermentation of step 2. In certain embodiments, the pH of the recycle stream may be adjusted, and further vitamins and metals added to supplement the stream.

In some embodiments, at least a portion of the fermentation broth is passed as a broth bleed from the first bioreactor to the second bioreactor. In some embodiments, an intermediate produced in the first bioreactor is removed as a permeate bleed. In some embodiments, the broth bleed and the permeate bleed are combined prior to their transfer to the second bioreactor. In some embodiments, the combined broth and permeate bleeds are processed to remove at least a portion of the intermediate prior to transfer to the second bioreactor.

In some embodiments, one or both of the microorganisms of step 1 and step 2 are wild type. In some embodiments, one or both of the microorganism of step 1 and step 2 are genetically modified. In some embodiments, the microorganism of step 1 is wild type, and the microorganism of step 2 is genetically modified. In some embodiments, the microorganism of step 1 is genetically modified, and the microorganism of step 2 is wild type.

In some embodiments, wherein the gaseous feedstock comprises CO, CO₂, H₂, or mixtures thereof, the culturing is conducted under anaerobic conditions. In other embodiments, wherein the gaseous C1 feedstock comprises CH₄, the culturing is operated under aerobic conditions. In some embodiments, both step 1 and step 2 are operated under anaerobic conditions. In some embodiments, both step 1 and step 2 are operated under aerobic conditions.

In some embodiments, CO₂ produced by a carbohydrate-fermenting microorganism (e.g., in step 2) is consumed by a C1-fixing microorganism (e.g., in step 1). In this embodiment, CO₂ produced by the carbohydrate-fermenting microorganism is a co-substrate for the C1-fixing microorganism.

In some embodiments, wherein the first microorganism is a C1-fixing microorganism and the second microorganism is a carbohydrate-fermenting microorganism, production of an intermediate by step 1 has a greater carbon efficiency than production of the intermediate by the process of step 2. In some embodiments, the amount of ATP generated by a carbohydrate-fermenting microorganism (e.g., in step 2) is greater than the amount of ATP generated by a C1-fixing microorganism (e.g., in step 1). In some embodiments, ATP generated by the carbohydrate-fermenting microorganism provides the energy required for the conversion of an intermediate to a final product in step 2.

As used herein, the term “step” can be interpreted to encompass one or more enzymatic conversions in a single microorganism. For example, in step 1, a first microorganism may convert “compound A” to “compound B” and “compound B” to “compound C,” and in step 2, a second microorganism may convert “compound C” (the intermediate) to “compound D” (the product). Exemplary intermediates and final products capable of being produced using the invention described herein can be found in Table 2.

TABLE 2
Potential Intermediates and Products
of Two-Step Fermentation Process.

| Intermediate | Product |
|---|---|
| Acetone | Isobutylene |
| β-hydroxyisovaleric acid/β- | Isobutylene |
| hydroxyisovalerate | |
| 3-hydroxybutyrate | 1,3-butanediol |
| Mevalonate | Isoprene, farnesene, pinene |
| | (isoprenoids) |
| 2-oxoglutarate | Succinate |
| Fatty acids | Long chained alcohols |
| Carboxylic acids | Alcohols |
| Fatty acids | Alkenes (O₂) |
| Fatty acids | Alkanes (O₂) |
| Hydroxy acids | Diols |
| Dicarboxylic acids | Diols |
| Chorismate | Vanillin |

In some embodiments, the intermediate, acetone, is produced by step 1, and the product, isobutylene is produced by step 2. Isobutylene, also referred to as isobutene or 2-methylpropene, is a major chemical building block with a market size of over 15 million tons and a global market value of $25-29 billion. Beyond its use in chemistry and as a fuel additive (15 Mt/yr), isobutylene may be converted to isooctane, a high performance, drop-in fuel for gasoline cars.

FIG. 3 shows a process according to one embodiment of the invention, wherein a pathway to isobutylene is provided in two process steps—a first step for the production of acetone, an intermediate in the pathway to isobutylene, and a second step for the conversion of the intermediate, acetone, to isobutylene. In accordance with the methods of the invention, the inventors identified the fermentation processes best metabolically suited to each of the process steps. In this example, acetone is produced in a C1-fixing fermentation process wherein a C1-containing substrate is converted to acetone by a culture comprising at least one C1-fixing microorganism. A carbon oxidation process is further provided for the conversion of the intermediate, acetone, to isobutylene. The carbon oxidation process oxidizes a carbohydrate feedstock and generates CO₂ and ATP. This generated ATP provides the energy requirement for the conversion of acetone to isobutylene, which is an energy-intensive step.

The process of FIGS. 3A and 3B can be performed in a single bioreactor, in which the first and second processes are operated under the same conditions. Alternatively, the process of FIG. 3 can be performed in a two-bioreactor system, as shown in FIG. 2, wherein the first process is operated under optimized C1-fixing process conditions, and the second process is operated under optimized carbon oxidation process conditions. When the processes are operated in a two-bioreactor system, at least a portion of CO₂ produced by the carbon oxidation process can be passed to the C1-fixing process for use as a co-substrate.

In some embodiments, a microorganism of the invention capable of producing isobutylene or an intermediate of isobutylene comprises one or more enzymes capable of converting i) acetyl-CoA to acetoacetyl-CoA, ii) acetoacetyl-CoA to acetoacetate, and/or iii) acetoacetate to acetone. See FIG. 3A. In some embodiments, the microorganism comprising one or more of these enzymes is a C1-fixing microorganism.

In some embodiments, a microorganism of the invention comprises a thiolase (thlA), such as an acetyl-coenzyme A acetyltransferase (EC 2.3.1.9), which is capable of converting acetyl-CoA to acetoacetyl-CoA; this step is shown in FIGS. 3A and 3B. This enzyme may also be referred to as acetyl-CoA acetyltransferase, acetyl-CoA:N-acetyltransferase, acetyl-CoA:acetyl-CoA C-acetyltransferase, β-keto-thiolase, acetoacetyl-CoA thiolase, β-acetoacetyl coenzyme A thiolase, 2-methylacetoacetyl-CoA thiolase, or 3-oxothiolase. Non-limiting examples of enzymes capable of converting acetyl-CoA to acetoacetyl-CoA include enzymes from Clostridium acetobutylicum (WP_010966157), Clostridium beijerinckii (WP_077869982), Clostridium kluyveri (WP_012104016, WP_012104014, WP_012104015), Cupriavidus necator (WP_011615089, WP_010810132), Escherichia coli (WP_000786547), Zoogloea ramigera (AAA27706.1), Candida tropicalis (XP 002547278), Clostridium carboxydivorans (EFG89502), Saccharomyces cerevisiae (NP_015297), and Clostridium tyrobutyricum.

In some embodiments, a microorganism of the invention comprises one or more CoA transferase enzymes, such as a heterodimer comprising acetoacetyl-CoA:acetate coenzyme A transferase A (cftA) and acetoacetyl-CoA:acetate coenzyme A transferase B (cftB). The CoA transferase (EC 2.8.3.9) is capable of converting acetoacetyl-CoA to acetoacetate and may also be referred to as butyrate-acetoacetate CoA-transferase, butyryl coenzyme A-acetoacetate coenzyme A-transferase, butyryl-CoA-acetoacetate CoA-transferase, or 3-oxoacid CoA-transferase. This step is shown in FIG. 3A. Non-limiting examples of cftA enzymes include enzymes from Clostridium acetobutylicum (WP_010890847), Clostridium beijerinckii (WP_012059996), Clostridium pasteurianum (WP_003447087), Escherichia coli (NP_416725), Clostridium saccharoperbutylacetonicum (WP_015395721), Acetoanaerobium sticklandii (WP_013361336), Clostridium botulinum (YP_001255345), Streptococcus pyogenes (NP_268526), Vibrio harveyi (WP_009698042), and Bacillus subtilis (WP_014477106). Non-limiting examples of cftB enzymes include enzymes from Clostridium acetobutylicum (WP_010890848), Clostridium beijerinckii (WP_012059997), Clostridium pasteurianum (WP_003447086), Escherichia coli (NP_416726), Clostridium saccharoperbutylacetonicum (WP_015395722), Acetoanaerobium sticklandii (WP_013361337), Clostridium botulinum (YP_001255346), Streptococcus pyogenes (NP_268527), Vibrio harveyi (WP_009698043), and Bacillus subtilis (WP_014477105). This step may also be catalyzed by a thioesterase (EC 3.1.2.20) or a phosphate butyryltransferase (EC 2.3.1.19) plus a butyrate kinase (EC 2.7.2.7); see, e.g. WO 2017/066498. In some embodiments, the enzyme capable of converting acetoacetyl-CoA to acetoacetate is a native enzyme. In some embodiments, the enzyme capable of converting acetoacetyl-CoA to acetoacetate is overexpressed.

In some embodiments, a microorganism of the invention comprises a decarboxylase, such as an acetoacetate decarboxylase (adc; EC 4.1.1.4), which is capable of converting acetoacetate to acetone. This step is shown in FIG. 3A. The acetoacetate decarboxylase may also be referred to as acetoacetic acid decarboxylase or acetoacetate carboxy-lyase. Non-limiting examples of acetoacetate decarboxylases include acetoacetate decarboxylases from Clostridium acetobutylicum (WP_010890849), Clostridium pasteurianum (KAJ50541), Clostridium saccharoperbutylacetonicum (WP_015395723), Clostridium beijerinckii (WP_012059998), Paenibacillus sp. (WP_053779363), Clostridium tetanomorphum (WP_051593393), Clostridium cellulolyticum, Chromobacterium violaceum (WP_011137067), Clostridium sp. BL-8 (WP_077860534), Clostridium diolis (WP_087702526), Bacillus subtilis (WP_012118395), Methanospirillum lacunae (WP_109969769), Sulfolobus islandicus (WP_012713251), and Aspergillus niger (CAK38022).

In some embodiments, the decarboxylase is an alpha-ketoisovalerate decarboxylase (kivd; EC 4.1.1.74) or a pyruvate decarboxylase (4.1.1.1). The alpha-ketoisovalerate decarboxylase may also be referred to as indolepyruvate decarboxylase, indol-3-yl-pyruvate carboxy-lyase, or 3-(indol-3-yl)pyruvate carboxy-lyase, and the pyruvate decarboxylase may also be referred to as alpha-carboxylase, pyruvic decarboxylase, alpha-ketoacid carboxylase, or 2-oxo-acid carboxy-lyase. Non-limiting examples of these decarboxylases include decarboxylases from Latococcus lactis (CAG34226), Zymomonas mobilis (AAA27697), Staphylococcus aureus (VEG29415), Schizosaccharomyces pombe (NP_001342796), Saccharomyces cerevisiae (NP_011601), and Fusarium sporotrichioides (PA0096).

In some embodiments, a microorganism of the capable of producing isobutylene or an intermediate of isobutylene comprises one or more enzymes capable of converting i) acetyl-CoA to acetoacetyl-CoA, ii) acetoacetyl-CoA to 3-hydroxybutyryl-CoA, iii) 3-hydroxybutyryl-CoA to 3-hydroxybutyrate, iv) 3-hydroxybutyrate to acetoacetate, and/or v) acetoacetate to acetone. In some embodiments, the enzyme capable of converting acetoacetyl-CoA to 3-hydroxybutyryl-CoA is a 3-hydroxybutyryl-CoA dehydrogenase (EC 1.1.1.157), an acetoacetyl-CoA reductase (EC 4.2.1.36), or an acetoacetyl-CoA hydratase (EC 4.2.1.119). In some embodiments, the enzyme capable of converting 3-hydroxybutyryl-CoA to 3-hydroxybutyrate is a thioesterase (EC 3.1.2.20) or a phosphate butyryltransferase (EC 2.3.1.19) plus a butyrate kinase (EC 2.7.2.7). In some embodiments, the enzyme capable of converting 3-hydroxybutyrate to acetoacetate is a 3-hydroxybutyrate dehydrogenase (EC 1.1.1.30). See, e.g., WO 2017/066498.

In some embodiments, a microorganism of the invention capable of producing isobutylene or an intermediate of isobutylene comprises one more enzymes capable of converting i) acetone to β-hydroxyisovaleric acid, ii) acetyl-CoA to 3-methyl-2-oxopentanoate, iii) 3-methyl-2-oxopentanoate to 3-methylbutanoyl-CoA, iv) 3-methylbutanoyl-CoA to 3-methylcrotonyl-CoA, v) 3-methylcrotonyl-CoA to β-hydroxyisovaleryl-CoA, vi) acetone to β-hydroxyisovaleryl-CoA, vii) β-hydroxyisovaleryl-CoA to β-hydroxyisovaleric acid, and/or viii) β-hydroxyisovaleric acid to isobutylene.

In some embodiments, a microorganism of the invention comprises a hydroxyisovalerate synthase, such as a hydroxymethylglutaryl-CoA synthase (HMG-CoA synthase) (EC 2.3.3.10), which is capable of converting acetone to β-hydroxyisovaleric acid. This step is shown in FIG. 3A. This enzyme may also be referred to as hydroxymethylglutaryl-CoA synthase, (S)-3-hydroxy-3-methylglutaryl-CoA acetoacetyl-CoA-lyase (CoA-acetylating), 3-hydroxy-3-methylglutaryl CoA synthetase, 3-hydroxy-3-methylglutaryl coenzyme A synthase, 3-hydroxy-3-methylglutaryl coenzyme A synthetase, 3-hydroxy-3-methylglutaryl-CoA synthase, 3-hydroxy-3-methylglutaryl-coenzyme A synthase, beta-hydroxy-beta-methylglutaryl-CoA synthase, acetoacetyl coenzyme A transacetase, hydroxymethylglutaryl coenzyme A synthase, or hydroxymethylglutaryl coenzyme A-condensing enzyme. Non-limiting examples of enzymes having hydroxyisovalerate synthase activity include enzymes from Pyrobaculum islandicum (WP_011762563), Haloferax volcanii (WP_004042308), Saccharomyces cerevisiae (NP 013580), Myxococcus xanthus (WP_011554270), Mus musculus (EDL18336), and Staphylococcus aureus (WP_000151982).

In some embodiments, a microorganism of the invention comprises enzymes involved in the conversion of acetyl-CoA to 3-methyl-2-oxopentanoate, which may include citramalate synthase (EC 2.3.1.182), 3-isopropylmalate dehydratase (EC 4.2.1.35), 3-isopropylmalate dehydrogenase (EC 1.1.1.85), acetolactate synthase (EC 2.2.1.6), ketol-acid reductoisomerase (EC 1.1.1.86), and/or dihydroxyacid dehydratase (EC 4.2.1.9). In some embodiments, the enzymes capable of converting acetyl-CoA to 3-methyl-2-oxopentanoate are native enzymes. In some embodiments, the enzymes capable of converting acetyl-CoA to 3-methyl-2-oxopentanoate are overexpressed.

In some embodiments, a microorganism of the invention comprises a ketoisovalerate oxidoreductase (EC 1.2.7.7), which converts 3-methyl-2-oxopentoate to 3-methylbutanoyl-CoA. In some embodiments, the ketoisovalerate oxidoreductase is a four-subunit enzyme, such as VorABCD from Methanothermobacter thermautotrophicus (WP_010876344.1, WP_010876343.1, WP_010876342.1, WP_010876341.1) or VorABCD from Pyrococcus furiosus (WP_011012106.1, WP_011012105.1, WP_011012108.1, WP_011012107.1).

In some embodiments, a microorganism of the invention comprises an enzyme that converts 3-methylbutanoyl-CoA to 3-methylcrotonyl-CoA. Non-limiting examples of enzymes having such activity include enzymes from Streptomyces avermitilis (AAD44196.1 or BAB69160.1) and Streptomyces coelicolor (AAD44195.1).

In some embodiments, a microorganism of the invention comprises an enzyme that catalyzes the conversion of 3-methylcrotonyl-CoA to β-hydroxyisovaleryl-CoA. This enzyme may be a crotonase/3-hydroxybutyryl-CoA dehydratase (EC 4.2.1.55), a crotonyl-CoA carboxylase-reductase (EC 1.3.1.86), a crotonyl-CoA reductase (EC 1.3.1.44), a 3-hydroxypropionyl-CoA dehydratase (EC 4.2.1.116), or an enoyl-CoA hydratase (4.2.1.17). Non-limiting examples capable of catalyzing this step include enzymes from Clostridium beijerinckii (ABR34202.1), Clostridium acetobutylicum (NP 349318.1), Myxococcus xanthus (WP_011553770.1), Treponema denticola (NP 971211.1), Euglena gracilis (AAW66853.1), Metallosphaera sedula (WP_012021928.1), and Bacillus anthraces (WP_000787371.1).

In some embodiments, a microorganism of the invention comprises a β-hydroxyisovaleryl-CoA synthase, which converts acetone to β-hydroxyisovaleryl-CoA.

In some embodiments, a microorganism of the invention comprises an enzyme that converts β-hydroxyisovaleryl-CoA to β-hydroxyisovaleric acid. This enzyme may be a thioesterase (EC 3.1.2.20) or a CoA-transferase, such as an acetyl-CoA:acetoacetyl-CoA transferase (EC 2.8.3.9). The enzyme that converts β-hydroxyisovaleryl-CoA to β-hydroxyisovaleric acid may also be referred to as acyl-CoA hydrolase, acyl coenzyme A thioesterase, acyl-CoA thioesterase, acyl coenzyme A hydrolase, thioesterase B, thioesterase II, or acyl-CoA thioesterase. Non-limiting examples of enzymes capable of converting β-hydroxyisovaleryl-CoA to β-hydroxyisovaleric acid include enzymes from Escherichia coli (WP_115207102), Bacillus subtilis (RUS06775), Escherichia coli (NP_414986), Staphylococcus aureus (VEE68738), Lactobacillus plantarum (POD89195), Handroanthus impetiginosus (PIN03838), Medicago truncatula (RHN70039), Trypanosoma cruzi (RNF22988), Pseudozyma hubeiensis SY62 (XP 012188138), Saccharomyces cerevisiae (P40353), and Picrophilus torridus (WP_011177594). This step may also be catalyzed by a phosphate butyryltransferase (EC 2.3.1.19) plus a butyrate kinase (EC 2.7.2.7); see, e.g. WO 2017/066498. In some embodiments, the enzyme capable of converting β-hydroxyisovaleryl-CoA to β-hydroxyisovaleric acid is a native enzyme. In some embodiments, the native enzyme capable of converting β-hydroxyisovaleryl-CoA to β-hydroxyisovaleric acid is overexpressed.

In some embodiments, a microorganism of the invention comprises an enzyme that converts β-hydroxyisovaleric acid to isobutylene, such as a hydroxyisovalerate phosphorylase/decarboxylase. This step is shown in FIGS. 3A and 3B. In some embodiments, the enzyme that converts β-hydroxyisovaleric acid to isobutylene may be a mevalonate diphosphate decarboxylase (EC 4.1.1.33), which may also be referred to as diphosphomevalonate decarboxylase, pyrophosphomevalonate decarboxylase, mevalonate-5-pyrophosphate decarboxylase, pyrophosphomevalonic acid decarboxylase, 5-pyrophosphomevalonate decarboxylase, mevalonate 5-diphosphate decarboxylase, or ATP:(R)-5-diphosphomevalonate carboxy-lyase. Non-limiting examples of mevalonate diphosphate decarboxylases include enzymes from Enterococcus faecium (CUX97682), Staphylococcus aureus (WP_123898653), Mus musculus (NP_619597), Bos taurus (NP_001068892), Homo sapiens (NP_002452), Zea mays (NP_001149256), Saccharomyces cerevisiae S288C (NP_014441), Schizosaccharomyces pombe (NP_594027), Aspergillus fumigatus (XP 752113), Trypanosoma brucei (XP 827840), Giardia lamblia (XP_001706526), Sulfodiicoccus acidiphilus (WP_126449008), and Metallosphaera sedula (WP_012021518). In other embodiments, the enzyme that converts β-hydroxyisovaleric acid to isobutylene may be a mevalonate 3-kinase (EC 2.7.1.185), which may also be referred to as ATP:(R)-MVA 3-phosphotransferase. Non-limiting examples of mevalonate 3-kinases include enzymes from Picrophilus torridus (Q6KZB1) and Thermoplasma acidophilum (Q9HIN1).

In some embodiments, a microorganism of the capable of producing isobutylene or an intermediate of isobutylene comprises one or more enzymes capable of converting i) acetyl-CoA to acetoacetyl-CoA, ii) acetoacetyl-CoA to 3-hydroxy-3-methyl-glutaryl-CoA, iii) 3-hydroxy-3-methyl-glutaryl-CoA to 3-methyl-glutaconyl-CoA, iv) 3-methyl-glutaconyl-CoA to 3-methylcrotonyl-CoA, v) 3-methylcrotonyl-CoA to β-hydroxyisovaleryl-CoA, vi) β-hydroxyisovaleryl-CoA to β-hydroxyisovaleric acid, and/or vii β-hydroxyisovaleric acid to isobutylene. See FIG. 3B. In some embodiments, the enzyme capable of converting acetyl-CoA to acetoacetyl-CoA is a thiolase, as described above. In some embodiments, the enzyme capable of converting acetoacetyl-CoA to 3-hydroxy-3-methyl-glutaryl-CoA is a hydroxymethylglutaryl-CoA synthase (EC 2.3.3.10). In some embodiments, the enzyme capable of converting 3-hydroxy-3-methyl-glutaryl-CoA to 3-methyl-glutaconyl-CoA is a methylglutaconyl-CoA hydratase (E.C. 4.2.1.18); an example of one such enzyme is from Acinetobacter sp. (ABA41462). In some embodiments, the enzyme capable of converting 3-hydroxy-3-methyl-glutaryl-CoA to 3-methyl-glutaconyl-CoA is 3-hydroxybutyryl-CoA dehydratase (E.C. 4.2.1.55); this enzyme may be from Myxococcus xanthus (WP_011553770), Pseudomonas putida (NP_744366 or WP_003250095), Pseudomonas (WP_003251320), or Yarrowia lipolytica (XP_500719). In some embodiments, the enzyme capable of converting 3-methyl-glutaconyl-CoA to 3-methylcrotonyl-CoA is a glutaconate CoA-transferase (2.8.3.12) or a methylcrotonoyl-CoA carboxylase (EC 6.4.1.14); non-limiting examples of such enzymes include enzymes from Myxococcus (WP_011554268 and WP_011554268) and Pseudomonas (WP_003113506 and WP_003100387). The enzyme capable of converting 3-methylcrotonyl-CoA to β-hydroxyisovaleryl-CoA may be an enoyl-CoA hydratase, such as an enzyme from Bacillus cereus (WP_000787370) or Metallosphaera sedula (WP_012021928) or a methylglutaconyl-CoA hydratase or 3-hydroxybutyryl-CoA dehydratase as described above.

In some embodiments, a microorganism of the invention capable of producing isobutylene or a precursor of isobutylene is engineered using the methods described in WO 2012/115527 or WO 2017/066498.

In some embodiments, a first microorganism comprises i) an enzyme capable of converting acetyl-CoA to acetoacetyl-CoA, ii) an enzyme capable of converting acetoacetyl-CoA to acetoacetate, and iii) an enzyme capable of converting acetoacetate to acetone. In some embodiments, the first microorganism is a C1-fixing microorganism, and acetone is the intermediate. In some embodiments, a second microorganism comprises i) an enzyme capable of converting acetone to β-hydroxyisovaleric acid and ii) an enzyme capable of comprising β-hydroxyisovaleric acid to isobutylene. In some embodiments, the second microorganism is a carbohydrate-fermenting microorganism, and isobutylene is the final product. In some embodiments, the first and second microorganisms are cultured in a single bioreactor. In other embodiments, the first and second microorganisms are cultured in two separate bioreactors, and the acetone produced by the first microorganism is passed from the first bioreactor to the second bioreactor. See FIG. 3A.

In some embodiments, a first microorganism comprises i) an enzyme capable of converting acetyl-CoA to acetoacetyl-CoA, ii) an enzyme capable of converting acetoacetyl-CoA to acetoacetate, iii) an enzyme capable of converting acetoacetate to acetone, and iv) an enzyme capable of converting acetone to β-hydroxyisovaleric acid. In some embodiments, the first microorganism is a C1-fixing microorganism, and β-hydroxyisovaleric acid is the intermediate. In some embodiments, a second microorganism comprises an enzyme capable of comprising β-hydroxyisovaleric acid to isobutylene. In some embodiments, the second microorganism is a carbohydrate-fermenting microorganism, and the final product is isobutylene. In some embodiments, the first and second microorganisms are cultured in a single bioreactor. In other embodiments, the first and second microorganisms are cultured in two separate bioreactors, and the β-hydroxyisovaleric acid produced by the first microorganism is passed from the first bioreactor to the second bioreactor.

In some embodiments, a first microorganism comprises i) enzymes capable of converting acetyl-CoA to 3-methyl-2-oxopentanoate, ii) an enzyme capable of converting 3-methyl-2-oxopentanoate to 3-methylbutanoyl-CoA, iii) an enzyme capable of 3-methylbutanoyl-CoA to 3-methylcrotonyl-CoA, iv) an enzyme capable of converting 3-methylcrotonyl-CoA to β-hydroxyisovaleryl-CoA, and v) an enzyme capable of converting β-hydroxyisovaleryl-CoA to β-hydroxyisovaleric acid. In some embodiments, first microorganism is a C1-fixing microorganism, and β-hydroxyisovaleric acid is the intermediate. In some embodiments, a second microorganism comprises an enzyme capable of comprising β-hydroxyisovaleric acid to isobutylene. In some embodiments, the second microorganism is a carbohydrate-fermenting microorganism, and the final product is isobutylene. In some embodiments, the first and second microorganisms are cultured in a single bioreactor. In other embodiments, the first and second microorganisms are cultured in two separate bioreactors, and the β-hydroxyisovaleric acid produced by the first microorganism is passed from the first bioreactor to the second bioreactor.

In some embodiments, a first microorganism comprises i) an enzyme capable of converting acetyl-CoA to acetoacetyl-CoA, ii) an enzyme capable of converting acetoacetyl-CoA to 3-hydroxy-3-methyl-glutaryl-CoA, iii) an enzyme capable of converting 3-hydroxy-3-methyl-glutaryl-CoA to 3-methyl-glutaconyl-CoA, iv) an enzyme capable of converting 3-methyl-glutaconyl-CoA to 3-methylcrotonyl-CoA, v) an enzyme capable of converting 3-methylcrotonyl-CoA to β-hydroxyisovaleryl-CoA, and vi) an enzyme capable of converting ii-hydroxyisovaleryl-CoA to β-hydroxyisovaleric acid. In some embodiments, first microorganism is a C1-fixing microorganism, and β-hydroxyisovaleric acid is the intermediate. In some embodiments, a second microorganism comprises an enzyme capable of comprising β-hydroxyisovaleric acid to isobutylene. In some embodiments, the first and second microorganisms are cultured in a single bioreactor. In other embodiments, the first and second microorganisms are cultured in two separate bioreactors, and the β-hydroxyisovaleric acid produced by the first microorganism is passed from the first bioreactor to the second bioreactor. See FIG. 3B.

FIG. 4 shows a process for the production of terpenes using a two-step process. The two-step process may involve a first step for the production of mevalonate, an intermediate in the pathway to terpene products, and a second step for the conversion of the intermediate, mevalonate, to a terpene. In accordance with the methods of the invention, the inventors identified the fermentation processes best metabolically suited to each of the process steps. In this example, mevalonate is produced in a C1-fixing fermentation process, wherein a C1-containing substrate is converted to mevalonate by a culture comprising at least one C1-fixing microorganism. A carbon oxidation process is further provided for the conversion of the intermediate product, mevalonate, to at least one terpene product. The carbon oxidation process oxidizes a carbohydrate feedstock and generates CO₂ and ATP. This generated ATP provides the energy requirement for the conversion of mevalonate to terpene(s).

The process of FIG. 4 can be performed in a single bioreactor, in which the first and second processes are operated under the same conditions. Alternatively, the process of FIG. 4 can be performed in a two-bioreactor system, as shown in FIG. 2, wherein the first process is operated under optimized C1-fixing process conditions, and the second process is operated under optimized carbon oxidation process conditions. When the processes are operated in a two-bioreactor system, at least a portion of CO₂ produced by the carbon oxidation process can be passed to the C1-fixing process for use as a co-substrate. In one embodiment, the terpene is selected from the group consisting of isoprene, farnesene, and isoprenoids. The isoprenoid may also be a hemiterpenoid (1 isoprene unit), a monoterpenoid (2 isoprene units), a sesquiterpenoid (3 isoprene units), a diterpenoid (4 isoprene units), a sesterterpenoid (5 isoprene units), a triterpenoid (6 isoprene units), a tetraterpenoid (8 isoprene units), or a polyterpenoid (9+ isoprene units).

In some embodiments, a mevalonate-producing microorganism comprises a thiolase (EC 2.3.1.9), an HMG-CoA synthase (EC 2.3.3.10), and an HMG-CoA reductase (EC 1.1.1.88). In some embodiments, the mevalonate-producing microorganism is a C1-fixing microorganism. In some embodiments, mevalonate produced by the C1-fixing microorganism is an intermediate in the production of isoprene or an isoprenoid such as farnesene by a carbohydrate-fermenting microorganism.

In some embodiments, an isoprene-producing microorganism comprises a mevalonate kinase (EC 2.7.1.36), a phosphomevalonate kinase (EC 2.7.4.2), a mevalonate diphosphate decarboxylase (EC 4.1.1.33), an isopentenyl-diphosphate delta-isomerase (EC 5.3.3.2), and an isoprene synthase (EC 4.2.3.27). In some embodiments, the isoprene-producing microorganism is a carbohydrate-fermenting microorganism. In some embodiments, mevalonate produced by a C1-fixing microorganism is converted to isoprene by a carbohydrate-fermenting microorganism.

In some embodiments, a farnesene-producing microorganism comprises a mevalonate kinase (EC 2.7.1.36), a phosphomevalonate kinase (EC 2.7.4.2), a mevalonate diphosphate decarboxylase (EC 4.1.1.33), a geranyltranstransferase Fps (EC:2.5.1.10), and a farnesene synthase (EC 4.2.3.46/EC 4.2.3.47). In some embodiments, the farnesene-producing microorganism is a carbohydrate-fermenting microorganism. In some embodiments, mevalonate produced by a C1-fixing microorganism is converted to farnesene by a carbohydrate-fermenting microorganism.

In some embodiments, a microorganism of the invention comprising one or more enzymes involved in the production of mevalonate, isoprene, or an isoprenoid is engineered using the methods and/or enzymes described in WO 2013/180584.

In some embodiments, mevalonate is toxic to a bacterium when not turned over quickly. Thus, in some embodiments, a mevalonate-producing microorganism (e.g., a C1-fixing microorganism) and a microorganism capable of converting mevalonate into a product (e.g., a carbohydrate-fermenting microorganism) are cultured in a single bioreactor to maximize turnover of mevalonate produced by the mevalonate-producing microorganism. In other embodiments, a mevalonate-producing microorganism and a microorganism capable of converting mevalonate into a product (e.g., a carbohydrate-fermenting microorganism) are cultured in separate bioreactors, and mevalonate produced in a first bioreactor is continuously passed to a second bioreactor.

FIGS. 5A and 5B show examples of a two-step fermentation process wherein a first microorganism is a carbohydrate-fermenting microorganism that produces an intermediate, and the second microorganism is a C1-fixing microorganism that converts the intermediate to a product. As shown in FIG. 5A, the intermediate may be a carboxylic acid or a dicarboxylic acid, and the product may be an alcohol or a diol. As shown in FIG. 5B, the intermediate may be a saturated or unsaturated fatty acid, and the product may be a saturated or unsaturated fatty alcohol. The processes shown in FIGS. 5A and 5B demonstrate utilization of ATP availability from the carbohydrate-fermenting step and reducing power of the C1-fixing step to produce a product.

The processes of FIGS. 5A and 5B can be performed in a single bioreactor, in which the first and second processes are operated under the same conditions. Alternatively, the processes of FIGS. 5A and 5B can be performed in a two-bioreactor system, as shown in FIG. 2, wherein the first step is operated under optimized carbon oxidation process conditions, and the second step is operated under optimized C1-fixing process conditions. When the processes are operated in a two-bioreactor system, at least a portion of CO₂ produced by the carbon oxidation process can be passed to the C1-fixing process for use as a co-substrate.

Various parameters may be manipulated during the fermentation of steps 1 and 2, including pH, nitrogen, media composition, and temperature. Rate of gas flow and rate of carbohydrate feedstock addition influence rate of production of an intermediate and rate of production of a final product. In some embodiments, rate of gas flow and rate of carbohydrate feedstock addition are optimized. In some embodiments, the rate of production of an intermediate by the first microorganism and the rate of production of a final product are optimized. In some embodiments, the rate of production of the intermediate by the first microorganism and the rate of production of the final product are of a similar order of magnitude to maximize the amount of final product that may be produced.

In some embodiments wherein step 1 and step 2 occur in a single bioreactor, the first microorganism may produce an essential component of the media for the second microorganism and/or the second microorganism may produce an essential component of the media for the first microorganism. For example, microorganism 1 may produce methionine, and microorganism 2 is a methionine auxotroph and/or microorganism 2 may produce thiamine, and microorganism 1 is a thiamine auxotroph.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A two-step fermentation method for producing a product, wherein the method comprises:
a. culturing a first microorganism under conditions wherein the first microorganism ferments a first feedstock to produce an intermediate; and
b. culturing a second microorganism under conditions wherein the second microorganism ferments a second feedstock to produce the product from the intermediate.

2. The method of claim 1, wherein the first feedstock or the second feedstock is a gaseous substrate.

3. The method of claim 2, wherein the gaseous substrate comprises one or more of CO, CO₂, H₂, and CH₄.

4. The method of claim 1, wherein the first feedstock or the second feedstock is a carbohydrate.

5. The method of claim 4, wherein the carbohydrate comprises one or more of xylose, arabinose, glucose, fructose, mannose, galactose, fucose, sucrose, maltose, melibiose, xylan, xylogluco-oligosaccharides, and mannitol.

6. The method of claim 1, wherein the first microorganism or the second microorganism is a C1-fixing microorganism.

7. The method of claim 6, wherein the C1-fixing microorganism is a member of a genus selected from the group consisting of Acetobacterium, Alkalibaculum, Blautia, Butyribacterium, Clostridium, Eubacterium, Moorella, Oxobacter, Sporomusa, and Thermoanaerobacter.

8. The method of claim 7, wherein the C1-fixing microorganism is derived from a parental bacterium selected from the group consisting of Clostridium autoethanogenum, Clostridium ljungdahlii, and Clostridium ragsdalei.

9. The method of claim 1, wherein the first microorganism or the second microorganism is a carbohydrate-fermenting microorganism.

10. The method of claim 9, wherein the carbohydrate-fermenting microorganism is selected from the group consisting of Escherichia coli, Bacillus subtilis, Caldicellulosiruptor saccharolyticus, Clostridium acetobutylicum, Clostridium beijerinckii, Lactococcus lactis, Lactobacillus, Klebsiella, Thermoplasma acidophilum, Picrophilus torridus, Zymomonas mobilis, Saccharomyces cerevisiae, Schizosaccharomyces pombe, Schwanniomyces (Debaryomyces) occidentalis, Kluyveromyces marxianus, and Yarrowia lipolytica.

11. The method of claim 1, wherein the first microorganism and the second microorganism are cultured in one bioreactor.

12. The method of claim 1, wherein the first microorganism is cultured in a first bioreactor to produce the intermediate, and the second microorganism is cultured in a second bioreactor to produce the product from the intermediate.

13. The method of claim 12, wherein at least a portion of an intermediate produced in the first bioreactor is passed to the second bioreactor.

14. The method of claim 1, wherein the intermediate is selected from the group consisting of acetone, β-hydroxyisovaleric acid, 3-hydroxybutyrate, mevalonate, 2-oxoglutarate, a fatty acid, a carboxylic acid, a dicarboxylic acid, a hydroxy acid, and chorismate, and the product is selected from the group consisting of isobutylene, 1,3-butanediol, isoprene, an isoprenoid, succinate, an alcohol, an alkane, an alkene, a diol, and vanillin.

15. The method of claim 14, wherein the intermediate is acetone or β-hydroxyisovaleric acid, and the product is isobutylene.

16. The method of claim 1, wherein:
a. the first microorganism is a C1-fixing microorganism and the first feedstock is a gaseous substrate;
b. the second microorganism is a carbohydrate-fermenting microorganism and the second feedstock is a carbohydrate substrate; and
c. the C1-fixing microorganism ferments the gaseous substrate to produce the intermediate, and the carbohydrate-fermenting microorganism ferments the carbohydrate substrate to produce the product from the intermediate.

17. The method of claim 16, wherein the intermediate is acetone or β-hydroxyisovaleric, and the product is isobutylene.

18. The method of claim 16, wherein:
a. the C1-fixing microorganism comprises one or more enzymes selected from the group consisting of an enzyme capable of converting acetyl-CoA to acetoacetyl-CoA, an enzyme capable of converting acetoacetyl-CoA to acetoacetate, and an enzyme capable of converting acetoacetate to acetone; and
b. the carbohydrate-fermenting microorganism comprises one or more enzymes selected from the group consisting of an enzyme capable of converting acetone to β-hydroxyisovaleric acid and an enzyme capable of converting β-hydroxyisovaleric acid to isobutylene.

19. The method of claim 16, wherein the carbohydrate-fermenting microorganism further produces CO₂.

20. The method of claim 19, wherein CO₂ produced by the carbohydrate-fermenting microorganism is a substrate for a C1-fixing microorganism.
